(19) United States
(12) Patent Application Publication
(10) Pub. No.: US 2021/0135131 A1
(43) Pub. Date: May 6, 2021

(54) PHOSPHORESCENT LIGHT-EMITTING COMPOUND

(71) Applicant: Sumitomo Chemical CO.,LTD, Tokyo (JP)

(72) Inventors: Kiran KAMTEKAR, Godmanchester (GB); William Tarran, Godmanchester (GB)

(73) Assignee: Sumitomo Chemical Company Limited, Tokyo (JP)

(21) Appl. No.: 17/255,781

(22) PCT Filed: Jun. 26, 2019
(86) PCT No.: PCT/GB2019/051804

§ 371 Date: Dec. 23, 2020

(30) Foreign Application Priority Data
Jun. 28, 2018 (GB) 1810665.8

(51) Int. Cl. H01L 51/00; C09K 11/06; C07F 15/00

(52) U.S. Cl. CPC H01L 51/0087; C09K 11/06; H01L 51/5016; C09K 2211/185; C09K 2211/1059; C07F 15/0093

(57) ABSTRACT

A phosphorescent light-emitting compound of formula (I): wherein: M is Pd(II) or Pt(II); Ar¹ is an aromatic or heteroaromatic group; R²—R⁴ in each occurrence is independently selected from the group consisting of: C₁₋₂₀ alkyl wherein one or more non-adjacent, non-terminal C atoms may be replaced with O, S, .CO or COO and one or more H atoms may be replaced with F; a group of formula (Ar²)p wherein p is at least 1 and Ar² in each occurrence is independently a C₆₋₂₀ aryl or a 5-20 membered heteroaryl which is unsubstituted or substituted with one or more substituents; and and R¹ is a group of formula (Ar²)P wherein at least one Ar² is a 6-membered heteroaromatic ring having C and N ring atoms. The compound of formula (I) may be used as a light-emitting material in a near infrared organic light-emitting device.

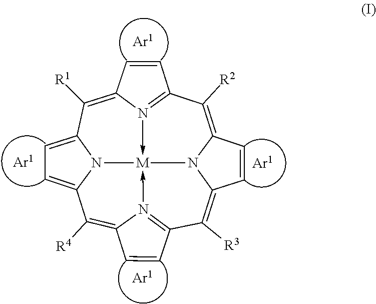

### FIELD

Embodiments of the present disclosure relate to phosphorescent light-emitting compounds, in particular near infra-red emitting compounds.

### BACKGROUND

Electronic devices containing active organic materials include devices such as organic light emitting diodes (OLEDs), organic photoresponsive devices (in particular organic photovoltaic devices and organic photosensors), organic transistors and memory array devices. Devices containing active organic materials can offer benefits such as low weight, low power consumption and flexibility. Moreover, use of soluble organic materials allows use of solution processing in device manufacture, for example inkjet printing or spin-coating.

An OLED includes an anode, a cathode and one or more organic layers between the anode and cathode including at least one organic light-emitting layer.

Holes are injected into the device through the anode and electrons are injected through the cathode during operation of the device. Holes in the highest occupied molecular orbital (HOMO) and electrons in the lowest unoccupied molecular orbital (LUMO) of a light-emitting material combine to form an exciton that releases its energy as light.

A light emitting layer may comprise a semiconducting host material and a light-emitting dopant wherein energy is transferred from the host material to the light-emitting dopant. For example, J. Appl. Phys. 65, 3610, 1989 discloses a host material doped with a fluorescent light-emitting dopant (that is, a light-emitting material in which light is emitted via decay of a singlet exciton), Phosphorescent dopants are also known (that is, light-emitting dopants in which light is emitted via decay of a triplet exciton).

OLEDs containing infrared emitting materials are also known as disclosed in, for example, Chuk-Lam Ho, Hua Li and Wai-Yeung Wong, “Red to near-infrared organometallic phosphorescent dyes for OLED applications”, J. Organomet. Chem. 751 (2014), 261-285 and Xiang et al, “Near-infrared phosphorescence: materials and applications”, Chem. Soc. Rev., 2013, 42, 6128.

WO 2012/034066 discloses a multichromophoric assembly comprising a metalloporphyrin. JP2011061095 discloses a tetrabenzoporphyrin semiconductor in which two meso-positions are substituted with a monovalent organic group.

WO 2013/168945 discloses a benzoporphyrin derivative and its use in an organic thin film transistor.

### SUMMARY

Near-infrared emitting materials have a relatively small bandgap compared to materials emitting in the visible region. Consequently, efficiency of infrared materials can be low due to a high proportion of excitons decaying non-radiatively in accordance with the energy gap law.

Furthermore, for certain applications, including but not limited to pulse oximetry, it is desirable for a near-infrared emitter to have a peak within a relatively narrow window within the broad (˜700-900 nm peak wavelength) near-infrared range.

The present inventors have found that substituting one or more meso-positions of certain phosphorescent metalloporphyrin compounds with a substituent containing a six-membered heteroaromatic group containing C and N ring atoms can allow for fine-tuning of the peak wavelength emitted by the compound, e.g. under electrical or light stimulation.

According to some embodiments, there is provided a compound of formula (I):

M is Pd(II) or Pt(II).

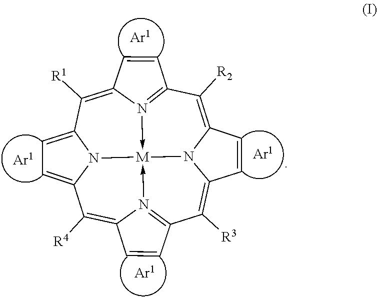

Ar¹ is an aromatic or heteroaromatic group which is unsubstituted or substituted with one or more substituents.

R²-R⁴ in each occurrence is independently selected from the group consisting of:
C₁₋₂₀ alkyl wherein one or more non-adjacent, non-terminal C atoms may be replaced with O, S, CO or COO and one or more H atoms may be replaced with F; and
a group of formula (Ar²)ₚ wherein p is at least 1 and AC in each occurrence is independently a C₆₋₂₀ aryl or a 5-20 membered heteroaryl which is unsubstituted or substituted with one or more substituents.

By “non-terminal C atom” of an alkyl group as used anywhere herein is meant a carbon atom of the alkyl group other than the methyl carbon of an n-alkyl group or each methyl carbon of a branched alkyl group.

R¹ is a group of formula (Ar²)ₚ wherein the Ar² (in the case where p=1) or at least one Ar² (in the case where p is greater than 1) is a 6-membered heteroaromatic ring having C and N ring atoms.

In some embodiments there is provided a composition comprising a host material and a phosphorescent light-emitting compound of formula (I).

In some embodiments there is provided a solution comprising a compound of formula (I) dissolved in one or more solvents.

In some embodiments there is provided an organic light-emitting device comprising an anode, a cathode and a light-emitting layer between the anode and cathode wherein the light-emitting layer comprises a compound of formula (I).

In some embodiments there is provided a method of forming an organic light-emitting device comprising the step of depositing a light-emitting layer comprising a compound of formula (I) over one of the anode and cathode, and depositing the other of the anode and cathode over the light-emitting layer.

### DESCRIPTION OF THE DRAWINGS

The disclosed technology and accompanying figures describe some implementations of the disclosed technology.

FIG. 1 illustrates an OLED according to some embodiments;

FIG. 2 is the photoluminescence spectra for a host-emitter composition according to an embodiment and two comparative compositions.

The drawings are not drawn to scale and have various viewpoints and perspectives. The drawings are some implementations and examples. Additionally, some components and/or operations may be separated into different blocks or combined into a single block for the purposes of discussion of some of the embodiments of the disclosed technology. Moreover, while the technology is amenable to various modifications and alternative forms, specific embodiments have been shown by way of example in the drawings and are described in detail below. The intention, however, is not to limit the technology to the particular implementations described. On the contrary, the technology is intended to cover all modifications, equivalents, and alternatives falling within the scope of the technology as defined by the appended claims.

### DETAILED DESCRIPTION

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof means any connection or coupling, either direct or indirect, between two or more elements; the coupling or connection between the elements can be physical, logical, electromagnetic, or a combination thereof. Additionally, the words “herein,” “above,” “below,” and words of similar import, when used in this application, refer to this application as a whole and not to any particular portions of this application. Where the context permits, words in the Detailed Description using the singular or plural number may also include the plural or singular number respectively. The word “or,” in reference to a list of two or more items, covers all of the following interpretations of the word: any of the items in the list, all of the items in the list, and any combination of the items in the list.

The teachings of the technology provided herein can be applied to other systems, not necessarily the system described below. The elements and acts of the various examples described below can be combined to provide further implementations of the technology. Some alternative implementations of the technology may include not only additional elements to those implementations noted below, but also may include fewer elements.

These and other changes can be made to the technology in light of the following detailed description. While the description describes certain examples of the technology, and describes the best mode contemplated, no matter how detailed the description appears, the technology can be practiced in many ways. Details of the system may vary considerably in its specific implementation, while still being encompassed by the technology disclosed herein. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the Detailed Description section explicitly defines such terms. Accordingly, the actual scope of the technology encompasses not only the disclosed examples, but also all equivalent ways of practicing or implementing the technology under the claims.

To reduce the number of claims, certain aspects of the technology are presented below in certain claim forms, but the applicant contemplates the various aspects of the technology in any number of claim forms.

In the following description, for the purposes of explanation, numerous specific details are set forth in order to provide a thorough understanding of implementations of the disclosed technology. It will be apparent, however, to one skilled in the art that embodiments of the disclosed technology may be practiced without some of these specific details.

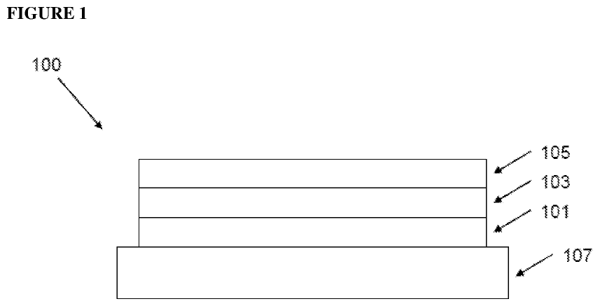

FIG. 1, which is not drawn to any scale, illustrates schematically an OLED 100 according to some embodiments. The OLED 100 may be carried on substrate 107. The OLED comprises an anode 101, a cathode 105 and a light-emitting layer 103 between the anode and the cathode.

As used herein, a layer “between” two other layers may be in direct contact with one or both of the other layers or may be spaced apart from one or both of the other layers by one or more intervening layers. Further layers (not shown) may be provided between the anode and the cathode including, without limitation, hole-transporting layers, electron-transporting layers, hole-blocking layers, electron-blocking layers, hole-injection layers and electron-injection layers.

Exemplary OLED structures including one or more further layers are, without limitation:

Anode/Hole-injection layer/Light-emitting layer/Cathode
Anode/Hole transporting layer/Light-emitting layer/Cathode
Anode/Hole-injection layer/Hole-transporting layer/Light-emitting layer/Cathode
Anode/Hole-injection layer/Hole-transporting layer/Light-emitting layer/Electron-transporting layer/Cathode
Anode/Hole-injection layer/Hole-transporting layer/Light-emitting layer/Electron-injecting layer/Cathode

Preferably, the device comprises one or both, more preferably both, of a hole-injection layer and a hole-transporting layer.

Preferably, the device comprises at least one of an electron-transporting layer and an electron injection layer.

Preferably, light-emitting layer 103 is the only light-emitting layer of the device.

Light-emitting layer 103 comprises a compound formula (I):

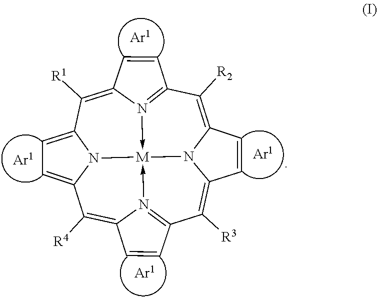

M may be Pd(II) or Pt(II), preferably Pt(II).

Ar¹ is an aromatic or heteroaromatic group which is unsubstituted or substituted with one or more substituents. Preferably, each Ar1 is benzene.

The compound of formula (I) may have formula (Ia):

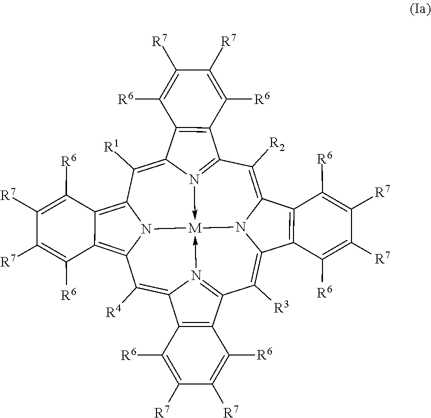

wherein each R⁶ and R is H or a substituent.

Preferably, each R⁶ is H.

Preferably, each R⁷ is independently H or a substituent. Substituents R⁷ may, independently in each occurrence, be selected from the group consisting of F, CN, NO₂ and C₁₋₂₀ alkyl wherein one or more non-adjacent, non-terminal C atoms may be replaced with O, S, CO or COO and one or more H atoms may be replaced with F.

In some embodiments, each R⁷ is H.

R¹ is a group of formula (Ar²)ₚ wherein p is at least 1 and Ar² in each occurrence is independently a C₆₋₂₀ aromatic group or a 5-20 membered heteroaromatic group which is unsubstituted or substituted with one or more substituents with the proviso that at least one Ar² is a 6-membered heteroaromatic ring having C and N ring atoms.

R²—R⁴ in each occurrence is independently selected from the group consisting of:
C₁₋₂₀ alkyl wherein one or more non-adjacent, non-terminal C atoms may be replaced with O, S, .CO or COO and one or more H atoms may be replaced with F; and a group of formula (Ar²)ₚ wherein p is at least 1 and Ar² in each occurrence is independently a C₆₋₂₀ aromatic group or a 5-20 membered heteroaromatic group which is unsubstituted or substituted with one or more substituents,

In some embodiments, one, two or all three of R²-R⁴ is a group R¹, i.e. a group of formula (Ar²)ₚ in which at least one Ar² is a 6-membered heteroaromatic ring having C and N ring atoms.

In some embodiments, none of R²-R⁴ is a group R¹.

Any group R²-R⁴ which is not a group R¹ is optionally and independently in each occurrence a C₁₋₄₀ hydrocarbyl group. Hydrocarbyl groups R²-R⁴ are optionally selected from C₁₋₂₀ alkyl and a group of formula (Ar²)ₚ wherein the or each Ar² is benzene which, independently in each occurrence, is unsubstituted or substituted with one or more C₁₋₁₂ alkyl groups. In some embodiments, each of R²-R⁴ is a group R¹ and R¹-R⁴ are the same.

The number of R¹ groups, the position of these groups and/or the structure of (Ar²)ₚ in these groups, may be selected to tune the peak emission wavelength of the compound of formula (I).

p may be 1-10, optionally 1-5.

Exemplary C₆₋₂₀ aryl groups Ar² are benzene and naphthalene which is unsubstituted or substituted with one or more substituents. 6-membered heteroaromatic groups Ar² having C and N ring atoms are optionally selected from: pyridine; 1,2-diazine, 1,3-diazine; 1,4-diazine; 1,2,3-triazine, 1,2,4-triazine; and 1,3,5 triazine, each of which is unsubstituted or substituted with one or more substituents.

Each Ar² is independently unsubstituted or substituted with one or more substituents. If present, substituents of Ar² may be selected from substituents R⁵ consisting of: F, CN, NO₂, and C₁₋₂₀ alkyl wherein one or more non-adjacent, non-terminal C atoms may be replaced with O, S, CO or COO and one or more FI atoms may be replaced with F.

In the case where p is greater than 2, the Ar² groups of (Ar²)ₚ, may be arranged in a linear or branching arrangement.

In a linear arrangement, each Ar² group is either a terminal Ar² group linked to only one other Ar² group or is a chain Ar² group directly linked to only two other Ar² groups. In the linear arrangement, (Ar²)ₚ contains only two terminal Ar² groups.

In a branched arrangement, at least one Ar² is a branching group which is bound to the porphyrin of formula (I) and at least two other Ar² groups, and/or at least one Ar² is a branching group which is bound to at least 3 other Ar² groups.

In some preferred embodiments, R¹ is a branching group of formula (II):

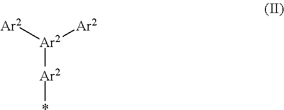

wherein * represents a bond to the porphyrin of formula (I).

In some embodiments, the Ar² group of R¹ which is bound directly to the porphyrin of formula (I) is a 6-membered heteroaromatic ring having C and N ring atoms. Optionally-according to these embodiments, p is 1.

In some embodiments the Ar² group bound directly to the porphyrin of the or each R¹ is not a 6-membered heteroaromatic ring having C and N ring atoms. Optionally according to these embodiments the (Ar²)ₚ group of R¹ comprises or consists of at least one C₆₋₂₀ arylene group, preferably at least one phenylene group, between the 6-membered heteroaromatic ring having C and N ring atoms and the porphyrin of formula (I).

Optionally, at least one of R¹-R⁴ is a group of formula (III):

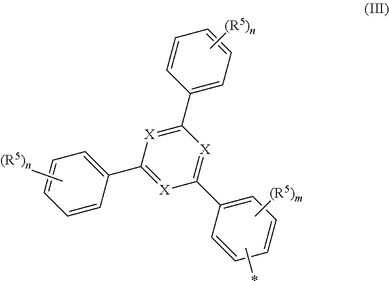

wherein * represents a bond; each X is independently selected from N and CR¹² wherein R¹² is H or a C₁₋₂₀ hydrocarbyl group; each R⁵ independently represents a substituent as described above; n is 0-5; and m is 0-4. A hydrocarbyl group R¹² may be selected from the group consisting of C₁₋₁₂ alkyl and phenyl which is unsubstituted or substituted with one or more C₁₋₁₂ alkyl groups.

In some embodiments, each of R¹-R⁴ is a group of formula (III).

Optionally, the group of formula (III) has formula (Ilia):

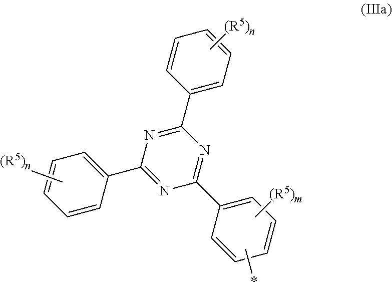

Exemplary compounds of formula (I) are:

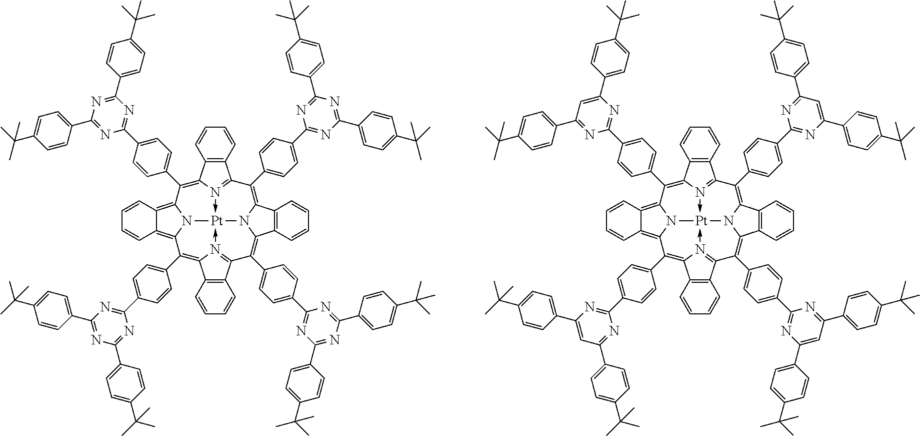

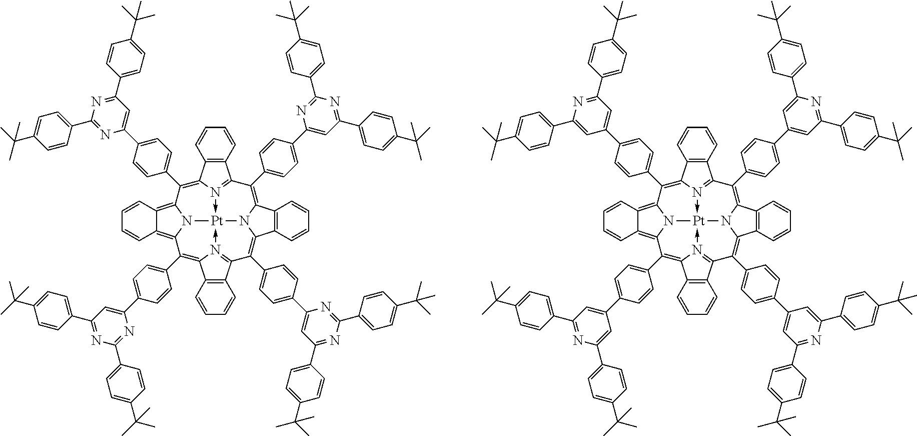

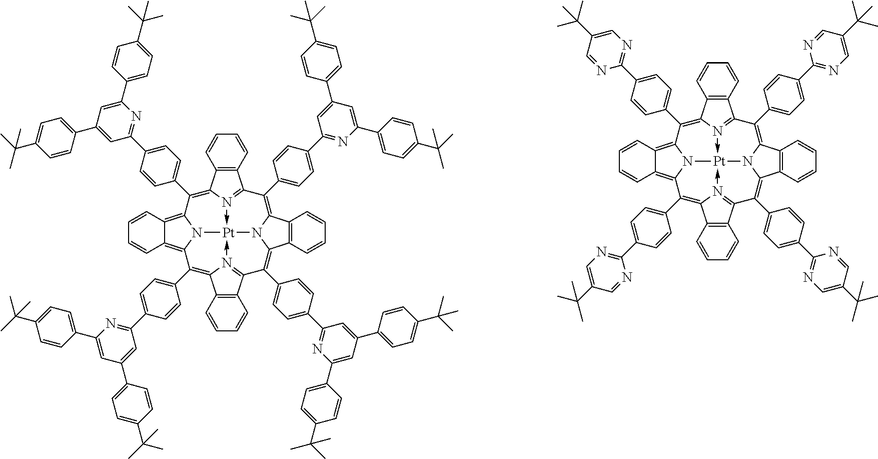

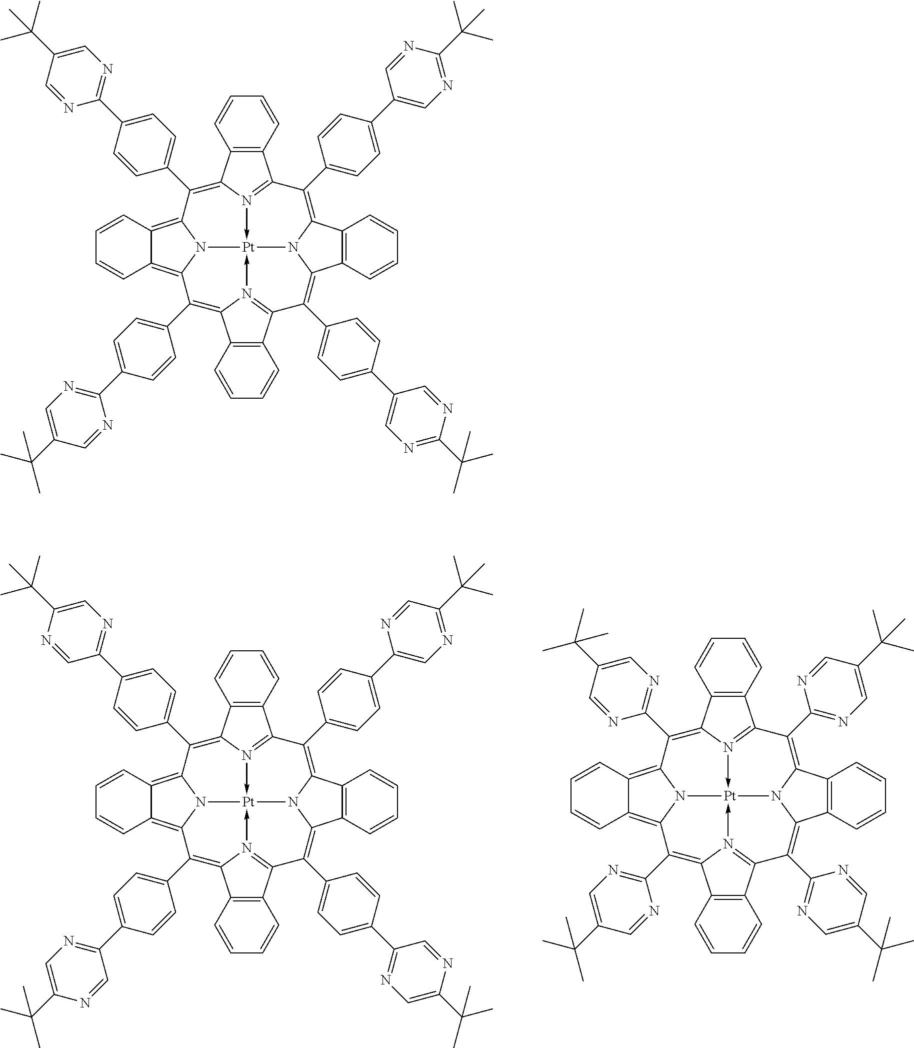

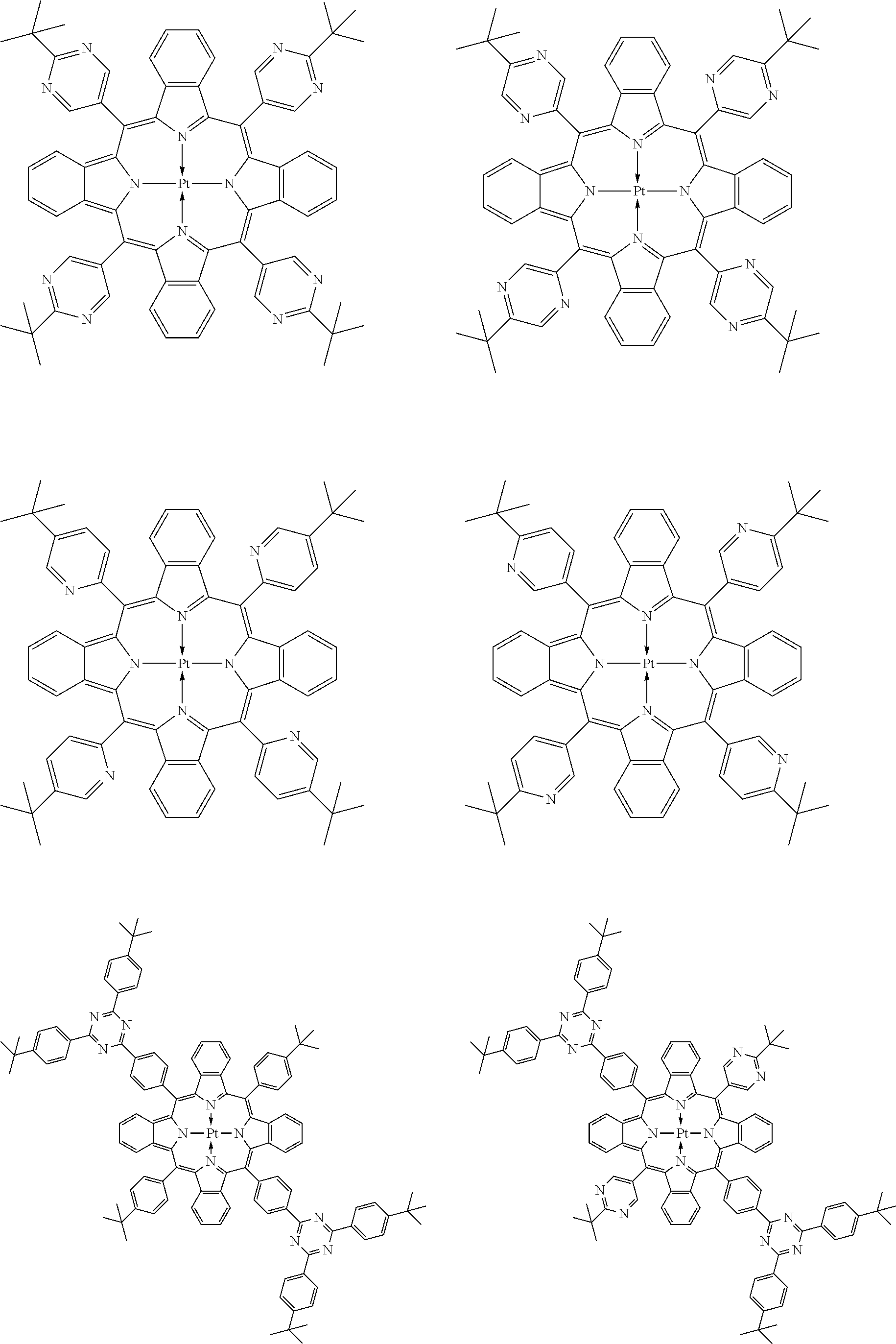

The compound of formula (I) is a phosphorescent compound. The compound of formula (I) preferably has a photoluminescent spectrum with a peak in the range of 700-900 nm, preferably 750-850 nm.

The photoluminescence spectrum of the compound of formula (I) may be measured by casting 5 wt % of the material in a polystyrene film onto a quartz substrate and measuring in a nitrogen environment using apparatus C9920-02 supplied by Hamamatsu.

Preferably, at least 90% or 95% of the light emitted by the device when in use, more preferably all light, is light emitted from the infrared emitter.

The compound of formula (I) may be used in combination with a host material having a triplet excited state energy level T₁ that is at least the same as or higher than the compound of formula (I) in order to allow transfer of triplet excitons from the host material to the phosphorescent compound of formula (I). Light-emitting layer 103 may comprise or consist of a host material and a compound of formula (I).

The triplet excited state energy levels of a host material and a phosphorescent compound may be determined from the energy onset of its phosphorescence spectrum measured by low temperature phosphorescence spectroscopy (Y. V. Romaovskii et al, Physical Review Letters, 2000, 85 (5), p 1027, A. van Dijken et al, Journal of the American Chemical Society, 2004, 126, p 7718).

The host material may be a polymer or a non-polymeric material.

The compound of formula (I) may be blended with or covalently bound to the host material.

The compound of formula (I) may be provided in an amount in the range of 0.1-40 wt % relative to the host in a composition comprising or consisting of a mixture of the host and the compound of formula (I).

In the case of a host polymer the compound of formula (I) may be provided as a side-group or end group of the polymer backbone or as a repeat unit in the backbone of the polymer. In this case, repeat units comprising a compound of formula (I) may form 0.1-40 mol % of the repeat units of the polymer.

A host polymer may comprise a repeat unit of formula (V):

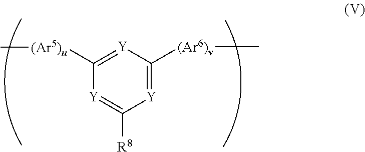

wherein Ar⁵ and Ar⁶ are each independently aryl or heteroaryl that may be unsubstituted or substituted with one or more, optionally 1, 2, 3 or 4, substituents; u and v in each occurrence is independently at least 1, optionally 1, 2 or 3, preferably 1; R⁸ is a substituent; and Y is N or CR⁹, wherein R⁹ is H or a substituent, preferably H or C₁₋₁₀ alkyl and with the proviso that at least one Y is N.

Preferably, Ar⁵ and Ar^{b} and are each independently unsubstituted or substituted C₆₋₂₀ aryl, more preferably C₁₀₋₂₀ aryl. Exemplary groups Ar⁵ and Ar⁶ are phenyl and naphthyl, preferably naphthyl.

Preferably, R⁸ is a C₁₋₂₀ alkyl group or a group of formula -(Ar⁷)w wherein Ar⁷ independently in each occurrence is an aryl or heteroaryl group that may be unsubstituted or substituted with one or more, optionally 1, 2, 3 or 4, substituents and w is at least 1, optionally 1, 2 or 3.

Preferably, each Ar⁷ is independently selected from unsubstituted or substituted phenyl, pyridyl, pyrimidine, pyrazine and triazine.

Substituents of Ar⁵, Ar⁶ and Ar⁷ may be selected from substituted or unsubstituted alkyl, optionally C₁₋₂₀ alkyl, wherein one or more non-adjacent, non-terminal C atoms may be replaced with O, S, C═O or —COO— and one or more H atoms may be replaced with F.

In one preferred embodiment, all 3 groups Y are N.

Preferably, u and v are each 1.

Preferably, w is 1, 2 or 3.

Exemplary repeat units of formula (V) have the following structures which may be unsubstituted or substituted with one or more substituents, preferably one or more C₁₋₂₀ alkyl groups:

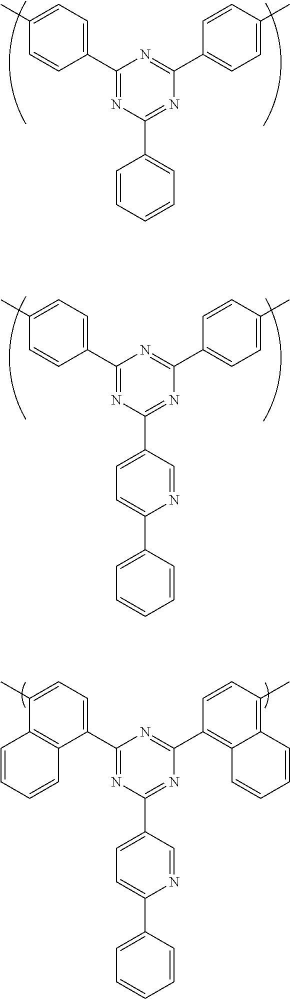

A host polymer may comprise a repeat unit of formula (XI);

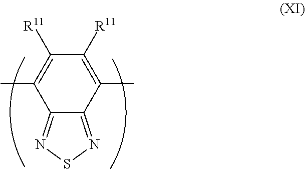

wherein each R¹¹ is independently H or a substituent. Optionally, substituents R¹¹ are independently selected from C₆₋₂₀ aryl that may be unsubstituted or substituted with one or more substituents, optionally one or more C₁₋₁₀ alkyl groups, and C₁₋₂₀ alkyl wherein one or more non-adjacent, non-terminal C atoms may be replaced with O, S, COO or CO and one or more H atoms may be replaced with F. Preferably, each R¹¹ is independently selected from H and C₁₋₂₀ alkyl.

A host polymer may comprise a repeat unit of formula (VI):

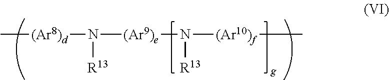

wherein Ar⁸, Ar⁹ and Ar^{1J} in each occurrence are independently selected from substituted or unsubstituted aryl or heteroaryl, g is 0, 1 or 2, preferably 0 or 1, R¹³ independently in each occurrence is a substituent, and d, e and f are each independently 1, 2 or 3.

R¹³, which may be the same or different in each occurrence when g is 1 or 2, is preferably selected from the group consisting of alkyl, optionally C₁₋₂₀ alkyl, Ar¹¹ and a branched or linear chain of Ar¹¹ groups wherein Ar¹¹ in each occurrence is independently substituted or unsubstituted aryl or heteroaryl.

Any two aromatic or heteroaromatic groups selected from Ar⁸, Ar⁹, and, if present, Ar¹⁰ and Ar¹¹ that are directly bound to the same N atom may be linked by a direct bond or a divalent linking atom or group. Preferred divalent linking atoms and groups include O, S; substituted N; and substituted C.

Ar⁸ and Ar¹⁰ are preferably C₆₋₂₀ aryl, more preferably phenyl, which may be unsubstituted or substituted with one or more substituents.

In the case where g=0, Ar⁹ is preferably C₆₋₂₀ aryl, more preferably phenyl, that may be unsubstituted or substituted with one or more substituents.

In the case where g=1, Ar⁹ is preferably C₆₋₂₀ aryl, more preferably phenyl or a polycyclic aromatic group, for example naphthalene, perylene, anthracene or fluorene, that may be unsubstituted or substituted with one or more substituents.

R¹³ is preferably Ar¹¹ or a branched or linear chain of Ar¹ groups. Ar¹¹ in each occurrence is preferably phenyl that may be unsubstituted or substituted with one or more substituents.

Exemplary groups R¹³ include the following, each of which may be unsubstituted or substituted with one or more substituents, and wherein * represents a point of attachment to N:

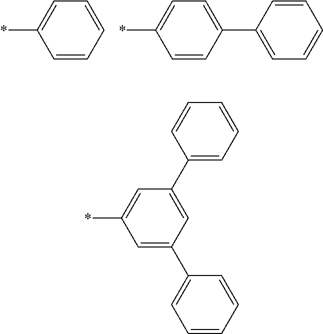

d e and f are preferably each 1.

Ar⁸, Ar⁹, and, if present, Ar¹⁰ and Ar¹¹ are each independently unsubstituted or substituted with one or more, optionally 1, 2, 3 or 4, substituents. Exemplary substituents may be selected from substituted or unsubstituted alkyl, optionally C₁₋₂₀ alkyl, wherein one or more non-adjacent, non-terminal C atoms may be replaced with optionally substituted aryl or heteroaryl (preferably phenyl), O, S, C═O or —COO— and one or more H atoms may be replaced with F.

Preferred substituents of Ar⁸, Ar⁹, and, if present, Ar¹⁰ and Ar¹¹ are C₁₋₄₀ hydrocarbyl, preferably C₁₋₂₀ alkyl.

Preferred repeat units of formula (VI) include unsubstituted or substituted units of formulae (VI-1), (VI-2) and (VI-3):

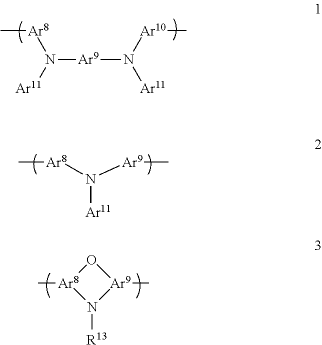

A host polymer may comprise arylene repeat units, preferably C₆₋₂₀ arylene repeat units, which may be unsubstituted or substituted with one or more substituents. Exemplary arylene repeat units are phenylene, fluorene, indenofluorene and phenanthrene repeat units, each of which may be unsubstituted or substituted with one or more substituents. Preferred substituents are selected from C₁₋₄₀ hydrocarbyl groups.

Arylene repeat units may be selected from formulae (VII)-(X):

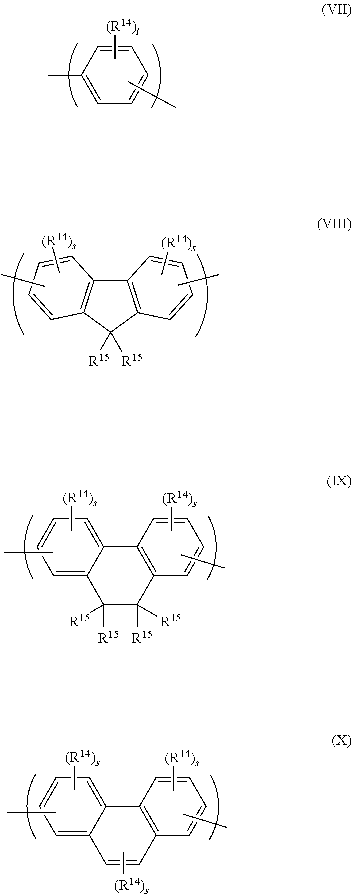

wherein t in each occurrence is independently 0, 1, 2, 3 or 4, preferably 1 or 2; R¹⁴ independently in each occurrence is a substituent; s in each occurrence is independently 0, 1 or 2, preferably 0 or 1; and R¹⁵ independently in each occurrence is a substituent wherein two R⁸ groups may be linked to form an unsubstituted or substituted ring.

Where present, each R¹⁴ and R¹⁵ may independently be selected from the group consisting of:
alkyl, optionally C₁₋₂₀ alkyl, wherein one or more non-adjacent, non-terminal C atoms may be replaced with optionally substituted aryl or heteroaryl, O, S, substituted N, C═O or —COO—, and one or more H atoms may be replaced with F;
aryl and heteroaryl groups, preferably C₆₋₂₀ aryl groups, more preferably phenyl, that may be unsubstituted or substituted with one or more substituents; and a linear or branched chain of aryl or heteroaryl groups, preferably C₆₋₂₀ aryl groups, more preferably phenyl, each of which groups may independently be substituted, optionally a group of formula —(Ar¹²)ᵣ wherein each Ar¹² is independently an aryl or heteroaryl group and r is at least 2, preferably a branched or linear chain of phenyl groups.

In the case where R¹⁴ or R¹⁵ comprises an aryl or heteroaryl group, or a linear or branched chain of aryl or heteroaryl groups, the or each aryl or heteroaryl group may be substituted with one or more substituents R⁸ selected from the group consisting of
alkyl, for example C₁₋₂₀ alkyl, wherein one or more non-adjacent, non-terminal C atoms may be replaced with O, S, substituted N, C═O and —COO— and one or more H atoms of the alkyl group may be replaced with F;
NR⁹₂, OR⁹, SR⁹, SiR⁹₃ and fluorine, nitro and cyano;
   wherein each R⁹ is independently selected from the group consisting of alkyl, preferably C₁₋₂₀ alkyl; and aryl or heteroaryl, preferably phenyl, optionally substituted with one or more C₁₋₂₀ alkyl groups.

Substituted N, where present, may be —NR¹⁰— wherein R¹⁰ is a substituent and is optionally a C₁₋₄₀ hydrocarbyl group, optionally a C₁₋₂₀ alkyl group.

Preferred substituents of aryl or heteroaryl groups of R¹⁴ or R¹⁵ are selected from C₁₋₂₀ alkyl.

In the case where two groups R¹⁵ form a ring, the one or more substituents of the ring, if present, are optionally selected from C₁₋₂₀ alkyl groups.

Preferably, each R¹⁴, where present, and R¹⁵ is independently selected from C₁₋₄₀ hydrocarbyl. Preferred C₁₋₄₀ hydrocarbyl groups are C₁₋₂₀ alkyl; unsubstituted phenyl; phenyl substituted with one or more C₁₋₂₀ alkyl groups; and a linear or branched chain of phenyl groups, wherein each phenyl may be unsubstituted or substituted with one or more C₁₋₂₀ alkyl groups.

A host polymer may comprise or consist of repeat units of formula (V), (VI) and/or (XI) and one or more arylene repeat units as described herein, optionally one or more arylene repeat units of formulae (VII)-(X).

Repeat units of formulae (V), (VI) and/or (XI) may each be provided in the host polymer in an amount in the range of 1-50 mol %, optionally 5-50 mol %.

Arylene repeat units may form 1-99 mol %, preferably 10-95 mol % of the repeat units of a host polymer.

Polymers as described herein including, without limitation, host polymers, may have a polystyrene-equivalent number-average molecular weight (Mn) measured by gel permeation chromatography in the range of about 1×10³ to 1×10⁸, and preferably 1×10³ to 5×10⁶. The polystyrene-equivalent weight-average molecular weight (Mw) of the polymers described herein may be 1×10³ to 1×10⁸, and preferably 1×10⁺ to 1×10⁷.

Polymers as described herein including, without limitation, host polymers, are preferably amorphous.

Charge Transporting and Charge Blocking Layers

A hole transporting layer may be provided between the anode of an OLED and a light-emitting layer containing a compound of formula (I).

An electron transporting layer may be provided between the cathode of an OLED and a light-emitting layer containing a compound of formula (I).

An electron blocking layer may be provided between the anode and the light-emitting layer.

A hole blocking layer may be provided between the cathode and the light-emitting layer.

Transporting and blocking layers may be used in combination. Depending on its HOMO and LUMO levels, a single layer may both transport one of holes and electrons and block the other of holes and electrons.

A charge-transporting layer or charge-blocking layer may be crosslinked, particularly if a layer overlying that charge-transporting or charge-blocking layer is deposited from a solution. The crosslinkable group used for this crosslinking may be a crosslinkable group comprising a reactive double bond such and a vinyl or acrylate group, or a benzocyclobutane group. The crosslinkable group may be provided as a substituent pendant from the backbone of a charge-transporting or charge-blocking polymer. Following formation of a charge-transporting or charge blocking layer, the crosslinkable group may be crosslinked by thermal treatment or irradiation.

If present, a hole transporting layer located between the anode and the light-emitting layer containing the compound of formula (I) preferably contains a hole-transporting material having a HOMO level of less than or equal to 5.5 eV, more preferably around 4.8-5.5 eV as measured by cyclic voltammetry. The HOMO level of the hole transporting material of the hole-transporting layer may be selected so as to be within 0.2 eV, optionally within 0.1 eV, of the compound of formula (I) in order to provide a small barrier to hole transport.

A hole-transporting material of a hole-transporting polymer may be a polymer comprising a repeat unit of formula (VI) as described herein, optionally a homopolymer of a repeat unit of formula (VI) or a copolymer comprising a repeat unit of formula (VI) and one or more co-repeat units, optionally one or more arylene co-repeat units as described herein. One or more repeat units of such a hole-transporting polymer may be substituted with a crosslinkable group, optionally a crosslinkable double bond group and/or a crosslinkable benzocyclobutane group, that may be crosslinked following deposition of the hole-transporting polymer to form the hole-transporting layer.

If present, an electron transporting layer located between the light-emitting layers and cathode preferably has a LUMO level of around 2.5-3.5 eV as measured by square wave cyclic voltammetry. A layer of a silicon monoxide or silicon dioxide or other thin dielectric layer having thickness in the range of 0.2-2 nm may be provided between the light-emitting layer nearest the cathode and the cathode.

An electron transporting layer may contain a polymer comprising a chain of optionally substituted arylene repeat units, such as a chain of fluorene repeat units.

HOMO and LUMO levels as described herein may be measured by cyclic voltammetry (CV) as follows.

The working electrode potential is ramped linearly versus time. When cyclic voltammetry reaches a set potential the working electrode's potential ramp is inverted. This inversion can happen multiple times during a single experiment. The current at the working electrode is plotted versus the applied voltage to give the cyclic voltammogram trace.

Apparatus to measure HOMO or LUMO energy levels by CV may comprise a cell containing a tert-butyl ammonium perchlorate/or tertbutyl ammonium hexafluorophosphate solution in acetonitrile, a glassy carbon working electrode where the sample is coated as a film, a platinum counter electrode (donor or acceptor of electrons) and a reference glass electrode no leak Ag/AgCl. Ferrocene is added in the cell at the end of the experiment for calculation purposes. (Measurement of the difference of potential between Ag/AgCl/ferrocene and sample/ferrocene).

Method and Settings:

3 mm diameter glassy carbon working electrode

Ag—AgCl/no leak reference electrode

Pt wire auxiliary electrode

0.1 M tetrabutylammonium hexafluorophosphate in acetonitrile

LUMO=4.8−ferrocene (peak to peak maximum average)+onset

Sample: 1 drop of 5 mg/mL in toluene spun @3000 rpm LUMO (reduction) measurement:

A good reversible reduction event is typically observed for thick films measured at 200 mV/s and a switching potential of −2.5V. The reduction events should be measured and compared over 10 cycles, usually measurements are taken on the 3 cycle. The onset is taken at the intersection of lines of best fit at the steepest part of the reduction event and the baseline.

Hole Injection Layers

A conductive hole injection layer, which may be formed from a conductive organic or inorganic material, may be provided between the anode and the light-emitting layer or layers to assist hole injection from the anode into the layer or layers of semiconducting polymer. A hole transporting layer may be used in combination with a hole injection layer.

Examples of doped organic hole injection materials include optionally substituted, doped poly(ethylene dioxy thiophene) (PEDT), in particular PEDT doped with a charge-balancing polyacid such as polystyrene sulfonate (PSS) as disclosed in EP 0901176 and EP 0947123, polyacrylic acid or a fluorinated sulfonic acid, for example Nation®; polyaniline as disclosed in U.S. Pat. Nos. 5,723,873 and 5,798,170; and optionally substituted polythiophene or poly(thienothiophene). Examples of conductive inorganic materials include transition metal oxides such as VOx, MoOx and RuOx as disclosed in Journal of Physics D: Applied Physics (1996), 29(11), 2750-2753.

Cathode

The cathode is selected from materials that have a work function allowing injection of electrons into the light-emitting layer or layers. Other factors influence the selection of the cathode such as the possibility of adverse interactions between the cathode and the light-emitting materials. The cathode may consist of a single material such as a layer of aluminium. Alternatively, it may comprise a plurality of metals, for example a bilayer of a low work function material and a high work function material such as calcium and aluminium as disclosed in WO 98/10621. The cathode may contain a layer containing elemental barium, for example as disclosed in WO 98/57381, Appl. Phys. Lett. 2002, 81(4), 634 and WO 02/84759 or a layer containing elemental magnesium. The cathode may contain a thin (e.g. 1-5 nm thick) layer of metal compound between the light-emitting layer(s) of the OLED and one or more conductive layers of the cathode, such as one or more metal layers. Exemplary metal compounds include an oxide or fluoride of an alkali or alkali earth metal, to assist electron injection, for example lithium fluoride as disclosed in WO 00/48258; barium fluoride as disclosed in Appl. Phys. Lett. 2001, 79(5), 2001; and barium oxide. In order to provide efficient injection of electrons into the device, the cathode preferably has a work function of less than 3.5 eV, more preferably less than 3.2 eV, most preferably less than 3 eV. Work functions of metals can be found in, for example, Michaelson, J. Appl. Phys. 48(11), 4729, 1977.

The cathode may be opaque or transparent. Transparent cathodes are particularly advantageous for active matrix devices because emission through a transparent anode in such devices is at least partially blocked by drive circuitry located underneath the emissive pixels. A transparent cathode comprises a layer of an electron injecting material that is sufficiently thin to be transparent. Typically, the lateral conductivity of this layer will be low as a result of its thinness. In this case, the layer of electron injecting material is used in combination with a thicker layer of transparent conducting material such as indium tin oxide.

It will be appreciated that a transparent cathode device need not have a transparent anode (unless, of course, a fully transparent device is desired), and so the transparent anode used for bottom-emitting devices may be replaced or supplemented with a layer of reflective material such as a layer of aluminium. Examples of transparent cathode devices are disclosed in, for example, GB 2348316.

Encapsulation

Organic optoelectronic devices tend to be sensitive to moisture and oxygen. Accordingly, the substrate 101 preferably has good barrier properties for prevention of ingress of moisture and oxygen into the device. The substrate is commonly glass, however alternative substrates may be used, in particular where flexibility of the device is desirable. For example, the substrate may comprise a plastic as in U.S. Pat. No. 6,268,695 which discloses a substrate of alternating plastic and barrier layers or a laminate of thin glass and plastic as disclosed in EP 0949850.

The device may be encapsulated with an encapsulant (not shown) to prevent ingress of moisture and oxygen. Suitable encapsulants include a sheet of glass, films having suitable barrier properties such as silicon dioxide, silicon monoxide, silicon nitride or alternating stacks of polymer and dielectric as disclosed in, for example, WO 01/81649 or an airtight container as disclosed in, for example, WO 01/19142. In the case of a transparent cathode device, a transparent encapsulating layer such as silicon monoxide or silicon dioxide may be deposited to micron levels of thickness, although in one preferred embodiment the thickness of such a layer is in the range of 20-300 nm. A getter material for absorption of any atmospheric moisture and/or oxygen that may permeate through the substrate or encapsulant may be disposed between the substrate and the encapsulant.

Solution Processing

Suitable solvents for forming solution processable formulations of the light-emitting compound of formula (I) and compositions thereof may be selected from common organic solvents, such as mono- or poly-alkylbenzenes such as toluene and xylene and mono- or poly-alkoxybenzenes, and mixtures thereof.

Exemplary solution deposition techniques for forming a light-emitting layer containing a compound of formula (I) include printing and coating techniques such spin-coating, dip-coating, roll-to-roll coating or roll-to-roll printing, doctor blade coating, slot die coating, gravure printing, screen printing and inkjet printing.

Coating methods, such as those described above, are particularly suitable for devices wherein patterning of the light-emitting layer or layers is unnecessary—for example for lighting applications or simple monochrome segmented displays.

The same coating and printing methods may be used to form other layers of an OLED including (where present) a hole injection layer, a charge transporting layer and a charge blocking layer.

Applications

An organic light-emitting diode as described herein may be used, without limitation, in night vision goggles, sensors including, without limitation, pulse oximeters, and CMOS chips. A sensor may comprise one or more OLED as described herein and at least one photodetector device, the or each photodetector device being configured to detect emission from the one more OLEDs. Optionally, the OLED of a sensor, preferably the OLED of a wearable sensor, has an operating voltage of no more than 5 V.

### EXAMPLES

### Compound Example 1

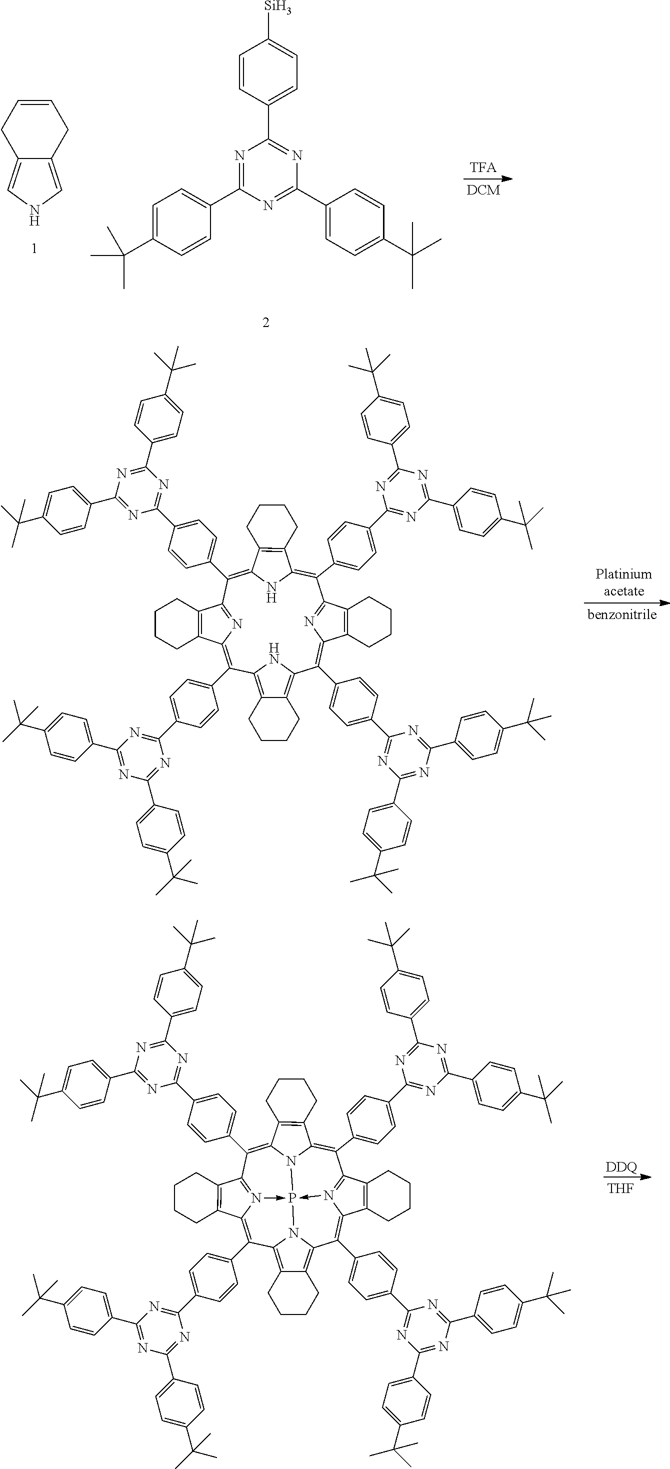

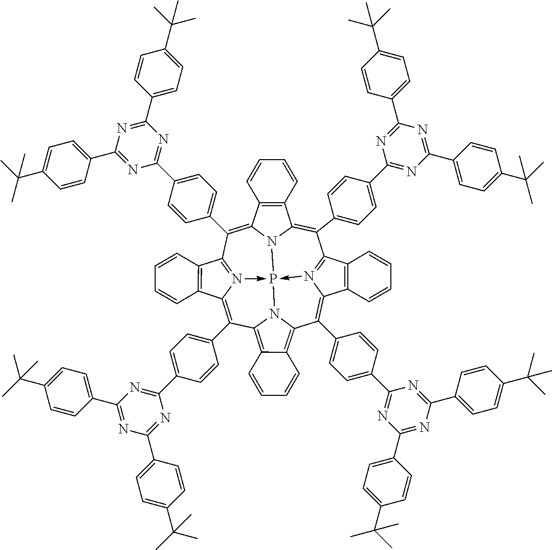

4,5,6,7-Tetrahydro-2H-isoindole can be synthesised as described in Chem. Mater., 2011, 23, 5296.

Stage 1

Starting material 1 (3.1 g, 25.5 mmol) and starting material 2 (11.4 g, 25.5 mmol) were dissolved in 2.2 L dichloromethane. Trifluoroacetic acid (1.74 g, 15.3 mmol) was added and the dark green reaction was stirred for 3 h at room temperature. 2,3-Dichloro-5,6-dicyano-1,4-benzoquinone (7.4 g, 32.6 mmol) was added and the reaction was stirred overnight at room temperature. Triethylamine (60 mL) was added and the reaction mixture was concentrated to ˜500 mL before being passed through a silica plug to remove baseline impurities. The filtrate as concentrated to yield a dark yellow solid. The solid was purified by column chromatography on silica (diameter 8 cm, height 80 cm) eluting with first hexanes, then dichloromethane and finally 1% methanol in dichloromethane to obtain a green solid which was recrystallized from methanol and used in the next step.

Stage 2

Platinum acetate (210 mg) was dissolved in benzonitrile (50 mL). Stage 1 material (800 mg) was added and the dark green solution was degassed for 1 h before being heated to 200° C. got 5 h. After cooling, then benzonitrile was removed by distillation and the dark red residue was purified by column chromatography on silica (diameter 2.2 cm, height 50 cm) eluting with 1-50% dichloromethane is hexane to isolate the Pt complex as a red solid (130 mg).

Stage 3

Stage 2 material (130 mg) was dissolved in THF (15 mL). 2,3-Dichloro-5,6-dicyano-1,4-benzoquinone (196 mL) was added and the reaction was heated to 70° C. for 2 h. After cooling the reaction was quenched with trimethylamine and concentrated. The crude material was purified by repeated column chromatography on silica (diameter 0.8 cm, height 40 cm) eluting with 20-100% dichloromethane in hexane followed by repeated recrystallisations from methanol to yield the product as a dark green solid.

### Composition Example 1

A composition of Host Polymer 1 (95 wt %) and Compound Example 1 (5 wt %) was formed by dissolving these compounds in mixed xylenes and spin-casting the film onto a quartz disk.

Photoluminescent peak values and photoluminescent quantum yield (PLQY) values were measured in an integrating sphere connected to Hamamatsu C9920-02 with a xenon lamp L8474 and a monochromator for choice of exact wavelength.

For the purpose of comparison, Composition Example 1 was compared to Comparative Compositions 1A and 1B which were prepared as described for Composition Example 1 except that Comparative Emitter 1 and Comparative Emitter 2, respectively, were used in placed of Compound Example 1.

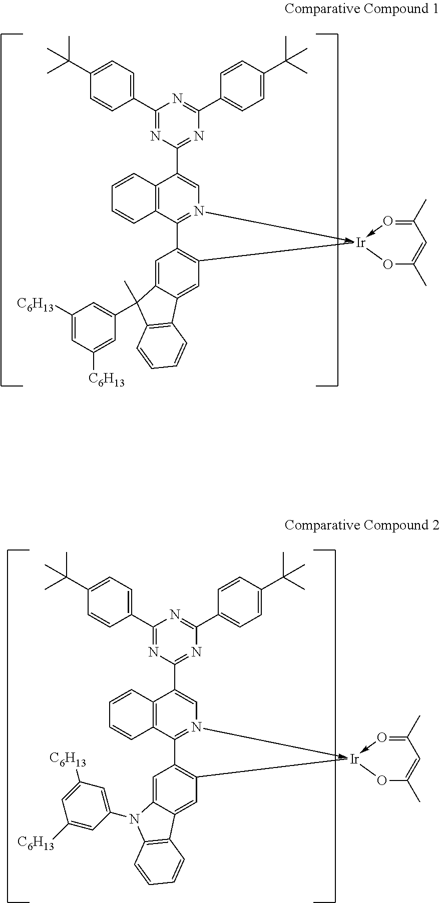

Comparative Compounds 1 and 2 are disclosed in WO 2017/103584, the contents of which are incorporated herein by reference.

Host Polymer 1 was formed by Suzuki polymerisation as disclosed in WO 00/53656 of the following monomers:

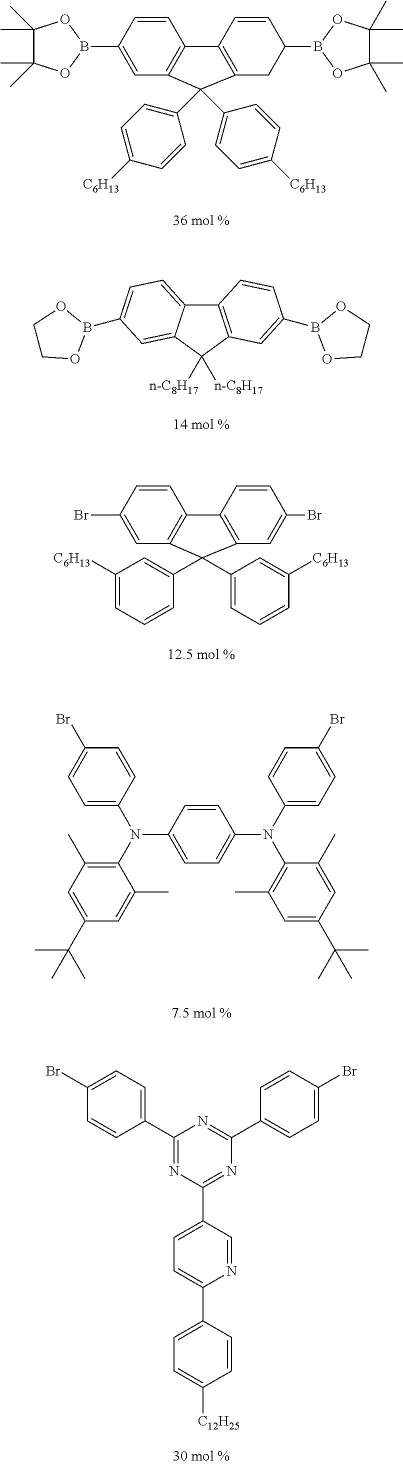

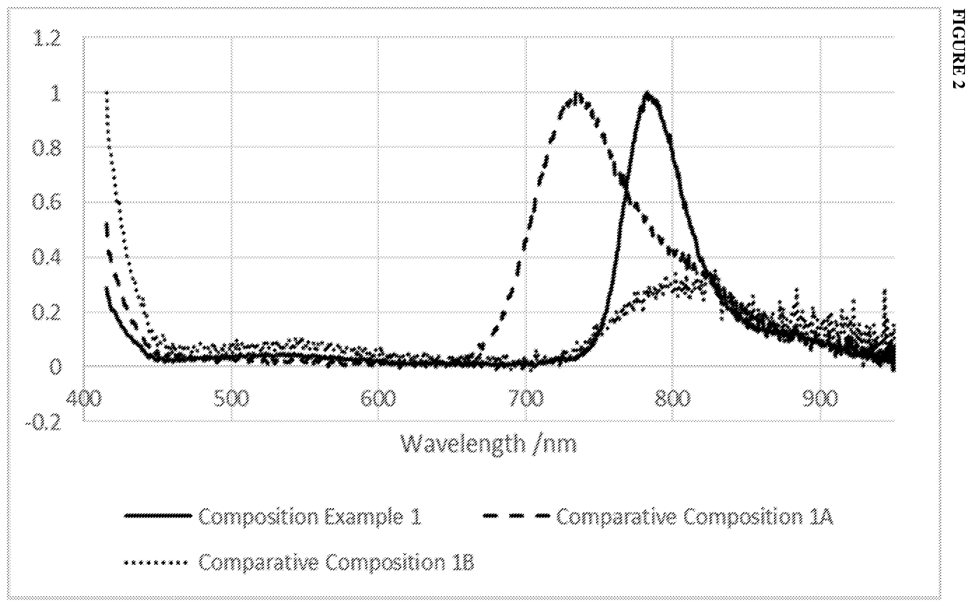

With reference to FIG. 2 and Table 1, although Comparative Composition 1A has a similar PLQY to that of Composition Example 1, it has a considerably shorter peak wavelength.

Infrared emitting materials have a relatively small bandgap compared to materials emitting in the visible region. Consequently, such materials can be susceptible to a high proportion of excitons decaying non-radiatively in accordance with the energy gap law, and yet Composition Example 1 has a PLQY comparable to that of Comparative Composition 1A despite its shorter peak wavelength.

Although Comparative Composition 1B has a similar peak wavelength to Composition Example 1, it has a much lower PLQY.

TABLE 1

| | | Emission peak |
|---|---|---|
| Composition | PLQY (%) | wavelength (nm) |
| Comparative Composition 1A | 25.6% | ~725 nm |
| Comparative Composition 1B | 4.4% | ~794 nm |
| Composition Example 1 | 21.5% | ~782 nm |

### Modelled Examples

Computer modelling of emission levels of modelled Compound Examples 2-5 was performed using Gaussian09 RevC.01 and compared with modelled Comparative Compound 3.

As set out in Table 2, the triphenyltriazine groups of Compound Examples 2-5 shift the emission peak to a longer wavelength, and the emission colour can be tuned by selection of number and/or position of triphenyltriazine substituents.

TABLE 2

| | | Number of | |
|---|---|---|---|
| | | triazine | QC modelled |
| Name | Structure | substituents | emission /nm |
| Comparative Compound 3 | 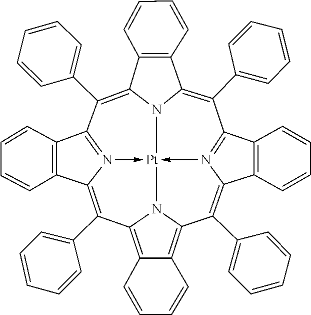 | 0 | 775 |
| Compound Example 2 | 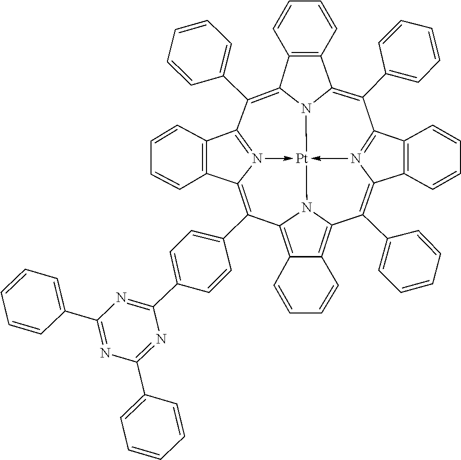 | 1 | 782 |
| Compound Example 3 | 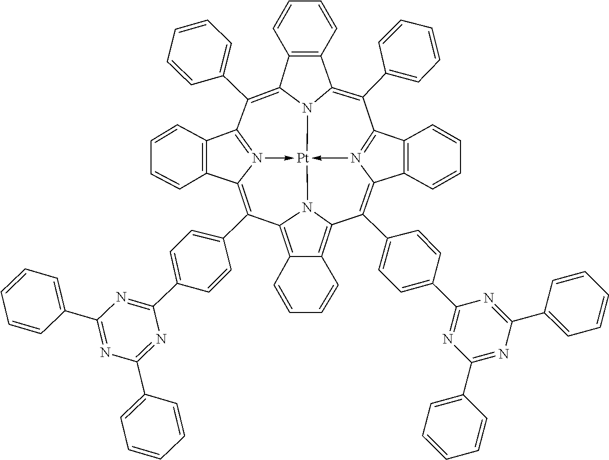 | 2 (adjacent) | 782 |
| Compound Example 4 | 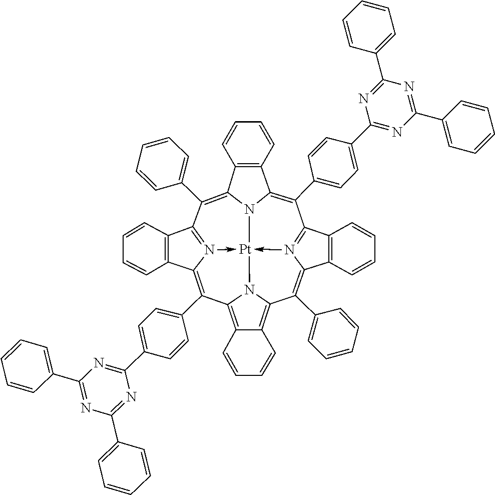 | 2 (opposite) | 789 |
| Compound Example 5 | 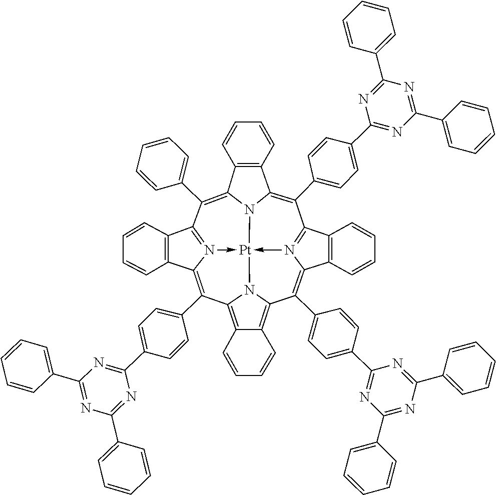 | 3 | 789 |
| Compound Example 6 | 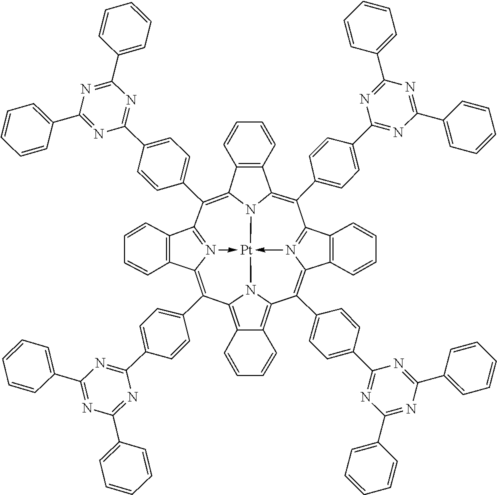 | 4 | 789 |

1. A phosphorescent light-emitting compound of formula (I): 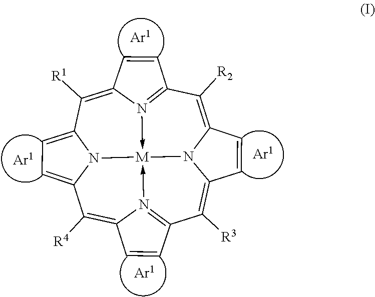
wherein:
M is Pd(II) or Pt(II);
Ar¹ is an aromatic or heteroaromatic group which is unsubstituted or substituted with one or more substituents;
R²-R⁴ in each occurrence is independently selected from the group consisting of:
C₁₋₂₀ alkyl wherein one or more non-adjacent, non-terminal C atoms may be replaced with O, S, .CO or COO and one or more H atoms may be replaced with F; and a group of formula (Ar²)ₚ wherein p is at least 1 and Ar² in each occurrence is independently a C₆₋₂₀ aryl or a 5-20 membered heteroaryl which is unsubstituted or substituted with one or more substituents;
and R¹ is a group of formula (Ar²)ₚ wherein at least one Ar² is a 6-membered heteroaromatic ring in which the only ring atoms are C and N ring atoms.

2. A phosphorescent light-emitting compound according to claim 1 wherein M is Pt (II).

3. A phosphorescent light-emitting compound according to claim 1 wherein each Ar¹ is a benzene ring which is unsubstituted or substituted with one or more substituents.

4. A phosphorescent light-emitting compound according to claim 1 wherein at least one of R²—R⁴ is a group of formula (Ar²)ₚ wherein at least one Ar² is a 6-membered heteroaromatic ring having C and N ring atoms.

5. A phosphorescent light-emitting compound according to claim 4 wherein each of R²—R⁴ is a group of formula (Ar²)ₚ wherein at least one Ar² of each of R²-R⁴ is a 6-membered heteroaromatic ring having C and N ring atoms.

6. A phosphorescent light-emitting compound according to claim 5 wherein R¹-R⁴ are the same.

7. A phosphorescent light-emitting compound according to claim 1 wherein each Ar² is independently selected from benzene, pyridine, 1,2-diazine, 1,3-diazine, 1,4-diazine, 1,2,3-triazine, 1,2,4-triazine and 1,3,5-triazine.

8. A phosphorescent light-emitting compound according to claim 1 wherein R¹ has formula (II):

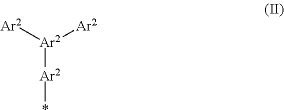

9. A phosphorescent light-emitting composition according to claim 1 wherein the 6-membered heteroaromatic ring having C and N ring atoms is triazine.

10. A phosphorescent light-emitting compound according to claim 1 wherein R¹ is a group of formula (III): 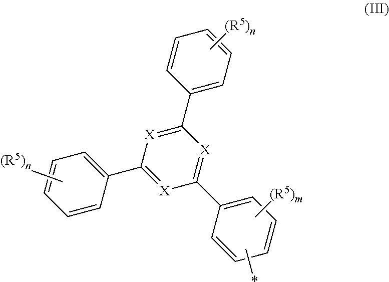
wherein * represents a bond; each R⁵ independently represents a substituent; n is 0-5; m is 0-4; and each X is independently selected from N and CR¹² wherein R¹² is H or a C₁₋₂₀ hydrocarbyl group and at least one X is N.

11. A phosphorescent light-emitting compound according to claim 10 wherein each of R¹-R⁴ is a group of formula (III).

12. A phosphorescent compound according to claim 1 wherein the compound has a photoluminescent spectrum with a peak in the range of 750-850 nm.

13. A composition comprising a host material and a phosphorescent light-emitting compound according to claim 1.

14. A solution comprising a compound according to claim 1 dissolved in one or more solvents.

15. An organic light-emitting device comprising an anode, a cathode and a light-emitting layer between the anode and cathode wherein the light-emitting layer comprises a compound according to claim 1.

16. A method of forming an organic light-emitting device according to claim 15 comprising the step of depositing the light-emitting layer over one of the anode and cathode, and depositing the other of the anode and cathode over the light-emitting layer.

17. A method according to claim 16 wherein the light-emitting layer is formed by depositing a solution comprising the compound dissolved in one or more solvents and evaporating the one or more solvents.

18. The solution according to claim 14 wherein the solution further comprises a host material.

19. The An organic light-emitting device according to claim 15 wherein the light-emitting layer further comprises a host material.
